(19) United States
(12) Patent Application Publication
(10) Pub. No.: US 2003/0069755 A1
(43) Pub. Date: Apr. 10, 2003

(54) System for devising treatment plans

(76) Inventors: Klaus Abraham-Fuchs, Erlangen (DE); Eva Rumpel, Erlangen (DE)

Correspondence Address: YOUNG & THOMPSON, 745 SOUTH 23RD STREET 2ND FLOOR, ARLINGTON, VA, 22202

(21) Appl. No.: 10/254,513

(22) Filed: Sep. 26, 2002

(30) Foreign Application Priority Data
Sep. 26, 2001 (DE) 101 47 470.9

(51) Int. Cl. G06F 17/60; A61B 5/00

(52) U.S. Cl. 705/2; 600/300

(57) ABSTRACT

A system for devising treatment plans, especially in the course of disease management services for patient information, instruction and motivation, possibly combined with telemonitoring of critical body values and, on this basis, early detection of risk situations, having a databank with a multiplicity of treatment modules stored in digital form, which can be interlinked with one another with the aid of operators to form a treatment plan.

### FIELD OF THE INVENTION

The invention relates to a system for devising treatment plans, especially in the course of disease management services for patient information, instruction and motivation, possibly combined with telemonitoring of critical body values and, on this basis, early detection of risk situations.

### BACKGROUND OF THE INVENTION

In the case of these disease management services, intensive use of the Internet is a medium-term aim, the present situation being that the patients are generally in communication with their disease management services provider (DMSP) by mail and—quite centrally—by telephone. However, expanding the service to cater for large numbers of patients requires further automation and consequently the use of suitable software. However, this software must be tailored not only to the disease in question but also to each individual service provider, since it is precisely the knowledge of structures and procedures of the respective disease management services provider and the resultant care concept where the core competences (and consequently also the competitive advantage) of these companies lie. Until now, these treatment plans have been manually devised, the sector using generally maintained software, which is not sector-specific, such as for example to support call centers.

U.S. Pat. 5,319,543 describes a workflow server for a medical recording system, concerned with improving the work flow in the electronic patient records and especially controlled access for various groups to the data of such electronic patient records. However, the system neither relates to a disease management service nor is concerned with the devising of an individual treatment plan for a specific patient.

In DE 195 23 036 A1 there is a description of an automatic program generator which concerns the creation of a user program for a computer, to be precise specifically a machine-code-based user program working with the source programs. The creation of such a user program on a source code basis is intended to be facilitated and made less individually dependent on the idiosyncrasies of the programmer by providing standard program parts in a semifinished state which are put together by the user to form a finished user program. However, this assisted creation of user programs for computers has nothing to do with a system for devising medical treatment plans.

### SUMMARY OF THE INVENTION

The invention is therefore based on the object of providing a system for the facilitated devising of treatment plans, in particular in the course of disease management services.

To achieve this object, a system of this type is characterized according to the invention by a databank with a multiplicity of treatment modules stored in digital form, which can be interlinked with one another with the aid of operators to form a treatment plan, a graphic user interface to assist the selection and assembly of the predetermined treatment. modules and also input devices for patient data for individualizing the treatment plan preferably being provided.

In a further refinement of the invention, it may be provided here that the treatment plan is drawn up in a machine-readable form and automatically performs tasks such as deadline management, record keeping, releasing information from the Internet, sending e-mails, designing forms for databanks, managing patient data and much more. Apart from the automation of these operations, the process knowledge mapped in software facilitates the in-house documentation of the operations and consequently forms the basis for quality management. In addition, the system facilitates transfer of the process knowledge to new employees or new areas of business.

To facilitate the assembly of the individual treatment modules to form a comprehensive treatment plan, it is possible according to a further feature of the present invention to provide that the databank additionally includes individually alterable standard treatment plans, in each case comprising a plurality of treatment modules for specific standard ailments. In this way it is made much easier to devise the treatment plan for many standard ailments, which indeed also frequently occur in practice, since all that is necessary is for individual modules to be additionally selected or incorporated in a different order to achieve an adaptation for the respective intended application or a quite specific patient.

The process steps of the treatment plan to be devised are selected from the given pool of possible substeps of the chronological order of later execution and are arranged into a module chain set up in chronological sequence, it being possible for the work flow to be both linear and in the form of a decision tree with decision nodes and/or loops.

To allow largely intuitive working with the system, it is possible here for command buttons and tool bars to be provided, with standard elements such as data entry (from master patient data or patient questionnaires), loops (for example annual subscription: for i<12 send monthly circular), branches and data outputs to be symbolically provided.

Quite generally, the arrangement is either set up in such a way that the desired modules are marked in a textual list or an arrangement of graphic elements (icons or the like) and are taken over into the process chain by a confirmatory mouse click or pressing of a key, or else that the desired modules—especially in the case of nonlinear decision trees—can be incorporated by “drag & drop”, that is to say dragging the marked module with the mouse or the like to the corresponding point of the decision tree.

As boundary values of the system, general medical knowledge (guidelines, standards) and/or company-specific knowledge (process knowledge, patient-related and/or cost-carrier-related target-group specific knowledge) may be included.

The use of predefined macros, including for example the supplementary “copy-paste” function for the speedy input of recurring partial structures, reduces the individual programming effort and, on the other hand, makes it possible to incorporate standards (such as for example the therapy guidelines of the specialist associations) into the company-own process management.

### BRIEF DESCRIPTION OF THE DRAWING

Further advantages, features and details of the invention emerge from the following description of an exemplary embodiment and on the basis of the drawing, in which:

FIG. 1 shows a flow plan of the system according to the invention for devising an individual treatment plan from predetermined modules,

FIG. 2 shows a representation of the possible automation of the operations within the individual treatment plan and

FIG. 3 shows a more detailed representation of the system for devising a specific treatment plan.

### DETAILED DESCRIPTION OF THE INVENTION

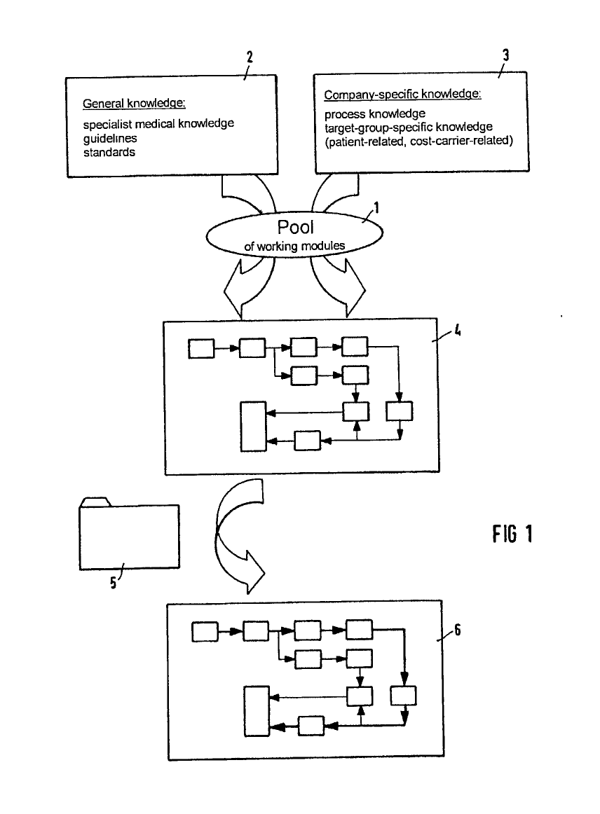

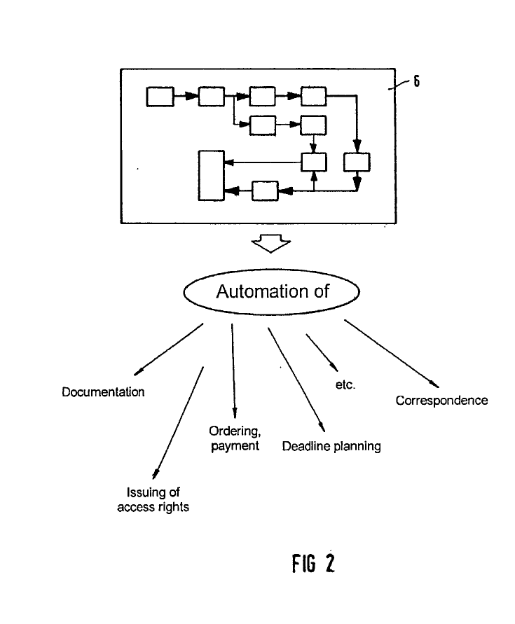

From a databank 1 of treatment modules, which is connected to a databank 2 concerning general medical knowledge and also guidelines and standards and also a databank 3 concerning company-specific knowledge, such as process knowledge or patient-related and cost-carrier-related target-group-specific knowledge, a general process management 4 can be devised by means of a graphic user interface and used as a basis for devising an individual treatment plan 6 by the input of patient data 5 via a corresponding input device. This individual treatment plan 6 from FIG. 1 makes it possible—as represented in FIG. 2—to automate both the documentation and the issuing of access rights for ordering and payment, deadline planning, correspondence etc.

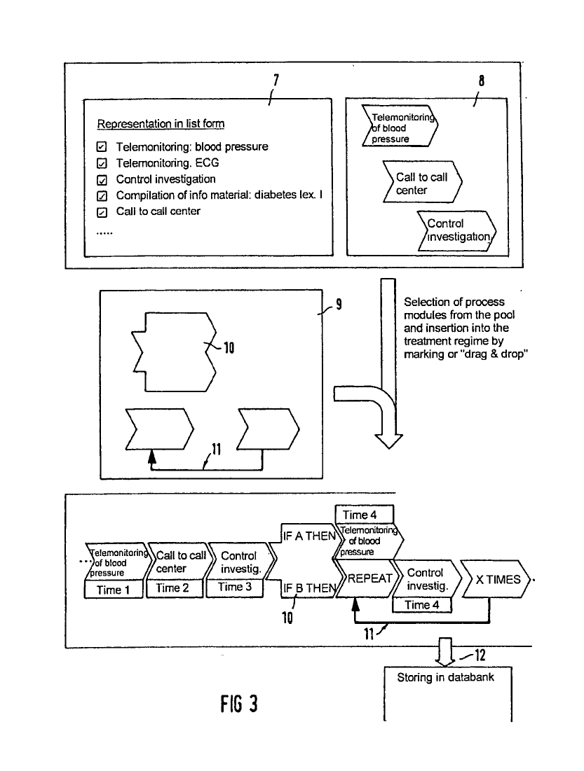

Included within the pool of working modules are individual modules which are shown as boxes in the general process management 4 or the treatment plan 6 and are represented (cf. in particular FIG. 3) either in the form of lists 7 or as graphic elements 8 and to which the graphic user interface allows access. By marking the module in the textual list 7 or in the arrangement of graphic elements 8 or else by “drag & drop”, the respectively marked modules can be incorporated at the correct point in the decision tree of the general process management 4, for example with the aid of a mouse click or pressing a key. In this case, the databank with the lists 7 or the graphic elements 8 of course also includes a databank 9 with control elements, to which the graphic user interface likewise offers access, in order either to form decision nodes 10 or loops 11, which can be operated at a corresponding point of the decision tree.

In the case of the very simplified exemplary embodiment shown in FIG. 3, it is intended for example at a time 1 (for example daily at 20:00 hours) for the ‘telemonitoring of blood pressure’ module to be called up, and, at a time 2, shortly after the time 1 or else at irregular intervals thereafter, for a call to be made to a call center, which in turn initiates at a time 3, that is for example every first week of the month, a control investigation. A decision node 10 can lead to the requirement at a time 4 either for a renewed telemonitoring of blood pressure or else repetition of a control investigation, it finally being possible via a loop 11 for this control investigation to be repeated as many predetermined times as desired. Finally, the storing of the treatment plan and its results in a memory databank 12 is indicated.

The individual treatment regime for a patient, shown at the bottom in FIG. 3, may be realized either by the doctor computer calling up the treatment plans of the patients or else the treatment plan for the individual patient spontaneously making itself known and, for example, “making an appointment” with the doctor computer.

The method according to the invention and the associated software tools assist a medical service provider in putting together an individual treatment plan for a patient in a very time-efficient way and, with the aid of the treatment plan stored in the computer, monitoring its implementation or executing it partly automatically. The treatment plan may in this case also comprise activities and decisions of all those involved in the process, for example the doctor, the patient, the care personnel of a call center looking after the patient or the doctor's receptionist.

The invention is not restricted to the exemplary embodiments represented. In particular, the system according to the invention is also suitable for devising treatment plans of all types in the medical context, including outside disease management services.

1. A system for devising treatment plans, in the course of disease management services for patient information, instruction and motivation, possibly combined with telemonitoring of critical body values and, on this basis, early detection of risk situations, the system comprising a databank having a multiplicity of treatment modules stored in digital form, which can be interlinked with one another with the aid of operators to form a treatment plan.

2. The system as claimed in claim 1, further comprising a graphic user interface to assist the selection and assembly of predetermined treatment modules.

3. The system as claimed in claim 1, further comprising input devices for patient data or an interface with electronic patient records (EPR) for individualizing the treatment plan.

4. The system as claimed in claim 1, wherein the treatment plan is drawn up in a machine-readable form and automatically performs tasks such as deadline management, record keeping, releasing information from the Internet, sending e-mails, designing forms for databanks, managing patient data or the like.

5. The system as claimed in claim 1, further comprising a further databank which additionally includes individually alterable standard treatment plans, comprising a plurality of treatment modules for specific standard ailments.

6. The system as claimed in claim 1, wherein that the work flow of the module chain is linear.

7. The system as claimed in claim 1, wherein the work flow of the module chain is formed as a decision tree with decision nodes and/or loops.

8. The system as claimed in claim 1, further comprising command buttons and tool bars to permit intuitive working.

9. The system as claimed in claim 1, wherein the desired modules are marked in a textual list or an arrangement of graphic elements (icons or the like) and are taken over into the process chain by a confirmatory mouse click or pressing of a key.

10. The system as claimed in claim 1, wherein the databank (1) can access a databank (2) concerning general medical knowledge and also guidelines and standards and/or a databank (3) concerning company-specific knowledge, such as process knowledge or patient-related and cost-carrier-related target-group-specific knowledge.

11. The system as claimed in claim 2, further comprising input devices for patient data or an interface with electronic patient records (EPR) for individualizing the treatment plan.

12. The system as claimed in claim 2, further comprising a further databank which additionally includes individually alterable standard treatment plans, comprising a plurality of treatment modules for specific standard ailments.

13. The system as claimed in claim 3, further comprising a further databank which additionally includes individually alterable standard treatment plans, comprising a plurality of treatment modules for specific standard ailments.

14. The system as claimed in claim 2, further comprising command buttons and tool bars to permit intuitive working.

15. The system as claimed in claim 3, further comprising command buttons and tool bars to permit intuitive working.

16. The system as claimed in claim 7, further comprising command buttons and tool bars to permit intuitive working.

17. The system as claimed in claim 11, further comprising command buttons and tool bars to permit intuitive working.

18. The system as claimed in claim 5, further comprising command buttons and tool bars to permit intuitive working.

19. The system as claimed in claim 12, further comprising command buttons and tool bars to permit intuitive working.

20. The system as claimed in claim 13, further comprising command buttons and tool bars to permit intuitive working.
